(19) United States
(12) Patent Application Publication
(10) Pub. No.: US 2018/0042452 A1
(43) Pub. Date: Feb. 15, 2018

(54) FLEXIBLE TUBE AND ENDOSCOPE INCORPORATING THE FLEXIBLE TUBE

(71) Applicant: OLYMPUS CORPORATION, Tokyo (JP)

(72) Inventors: Hiromitsu OKADA, Hachioji-shi (JP); Kohei ARAKI, Hachioji-shi (JP)

(73) Assignee: OLYMPUS CORPORATION, Tokyo (JP)

(21) Appl. No.: 15/783,545

(22) Filed: Oct. 13, 2017

Related U.S. Application Data
(63) Continuation of application No. PCT/JP2016/061153, filed on Apr. 5, 2016.

(30) Foreign Application Priority Data
May 20, 2015 (JP) 2015-102748

(51) Int. Cl. A61B 1/008; G02B 23/24; A61B 1/01

(52) U.S. Cl. CPC A61B 1/008; A61B 1/01; G02B 23/2476; A61B 1/00071

(57) ABSTRACT

A spiral tube that forms a flexible tube used for an insertion section is formed by spirally and densely winding a band-like element wire, and being coated by an external layer from the outer periphery side. In the element wire, adjustment portions are arranged in intervals that are greater than half the circumference of the spiral along the longitudinal direction of the band. A plurality of retaining portions are formed as holes or bottomed holes in a continuous manner. The retaining portions are shifted in the circumferential direction of the longitudinal axis so that the retaining portions that are formed in the adjacent portions of the spirally wound element wire would not be next to each other in the direction parallel to the longitudinal axis.

### CROSS-REFERENCE TO RELATED APPLICATIONS

This is a Continuation Application of PCT Application No. PCT/JP2016/061153, filed Apr. 5, 2016, which was published under POT Article 21(2) in Japanese.

This application is based upon and claims the benefit of priority from the Japanese Patent Application No. 2015-102748, filed May 20, 2015, the entire contents of which are incorporated herein by reference.

### BACKGROUND OF THE INVENTION

### 1. Field of the Invention

The present invention relates to a flexible tube that is provided in an insertion section, is freely bendable, and exhibits excellent insertability and resilience, as well as an endoscope incorporating such a flexible tube.

### 2. Description of the Related Art

Generally, in the use of an endoscope, its elongated insertion section is inserted into a lumen or body cavity that has flexures. The insertion section includes a distal end portion on the distal side of the insertion, a bendable portion continuous with the proximal side of the distal end portion, and a flexible tube continuous with the bendable portion and connected to an operation section of the endoscope.

At the time of insertion into a lumen, the flexible tube is inserted sequentially from the bendable portion. It is configured to be bent suitably in accordance with the flexures inside the lumen, and at the same time serves as a conveyor of propulsion to the inserted distal end portion. As part of the structure that realizes the flexibility, a spiral tube is provided inside a flexible tube, which has been known and is disclosed in Reference 1, Jpn. Pat. Appln. KOKAI Publication Nor 2012-120573 This spiral tube is configured by densely and spirally winding an elongated thin and narrow metal plate (element wire) into what is called a densely-wound portion so as not to create any space between the turns. The stricture exhibits flexibility, and with an initial tension applied, the structure also exhibits resilience.

### BRIEF SUMMARY OF THE INVENTION

According to an embodiment of the present invention, there is provided a flexible tube comprising: a spiral tube configured by densely winding a band-like element wire into a spiral form and applying an initial tension to the element wire along a direction of a longitudinal axis of the spiral form; and an external layer that covers an external periphery of the spiral tube, wherein a plurality of sets of an adjustment portion and a retaining portion are formed in the element wire in a continuous manner in a longitudinal direction of the element wire, each of adjustment portions including the adjustment portion is arranged between a pair of edge portions provided in a width direction of the element wire and configured to adjust a distance between the pair of edge portions in accordance with an external force applied, and each of retaining portions including the retaining portion is configured to retain the distance between the pair of edge portions, and the retaining portions formed in adjacent portions of the element wire when being wound are shifted in a peripheral direction of the longitudinal axis so as not to be positioned continuously in a direction. parallel to the longitudinal axis.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing an outer appearance of an endoscopic main body according to the first embodiment.

FIG. 2 is a sectional view of the flexible tube that is used in the insertion section according to the present embodiment.

FIG. 3A is a diagram showing an outer appearance of an element wire that forms a spiral tube when viewed from above.

FIG. 3B is a diagram showing an outer appearance of the spiral tube prepared by winding the element wire.

FIG. 3C is a diagram showing the positional relationship of inter-hole portions at the first pitch and the second pitch.

FIG. 3D is a conceptual diagram of the sectional structure of the spiral tube when viewed from the direction of the longitudinal axis.

FIG. 4A is a diagram for showing the outer appearance of the spiral tube in the straight state.

FIG. 4B is a diagram for showing the spiral tube in the bent state.

FIG. 5A is a conceptual diagram of the outer appearance of a spiral tube in the straight state.

FIG. 5B is a diagram showing part of the structure of a hole in one pitch of the spiral tube.

FIG. 5C is a conceptual diagram of the spiral tube in the state of being bent into a desired bending radius R1.

FIG. 5D is a conceptual diagram of the spiral tube in the state of being bent to the degree that the two walls of the hole are brought into a contact.

FIG. 6 is a conceptual diagram showing the positions of the inter-hole portions and the spiral tube that is in the bent state.

FIG. 7A is a diagram illustrating an outer appearance of the element wire that forms a flexible tube according to the second embodiment.

FIG. 7B is a conceptual diagram of the arrangement of inter-hole portions viewed from the direction of the longitudinal axis.

FIG. 8A is a diagram showing the shape of holes according to the first modification.

FIG. 8B is a diagram showing the shape of holes according to the second modification.

FIG. 9 is a diagram illustrating an outer appearance of an element wire that forms a flexible tube according to the third embodiment.

FIG. 10A is a cross section. of an element wire having a rectangular shape, as the first example of the fourth embodiment.

FIG. 10B is a cross section of an element wire having a convex curve and a concave curve on the shorter sides, as the second example of the fourth embodiment.

FIG. 10C is a cross section of an element wire having a pointed end surface and an indented end surface, as the third example of the fourth embodiment.

FIG. 10D is a cross section of an element wire having a through-hole, which is the fourth example of the fourth embodiment.

FIG. 10E is a cross section of an element wire having an adjustment portion, as the fifth example of the fourth embodiment.

### DETAILED DESCRIPTION OF THE INVENTION

Embodiments of the present invention will be described below with reference to the accompanying drawings.

### First Embodiment

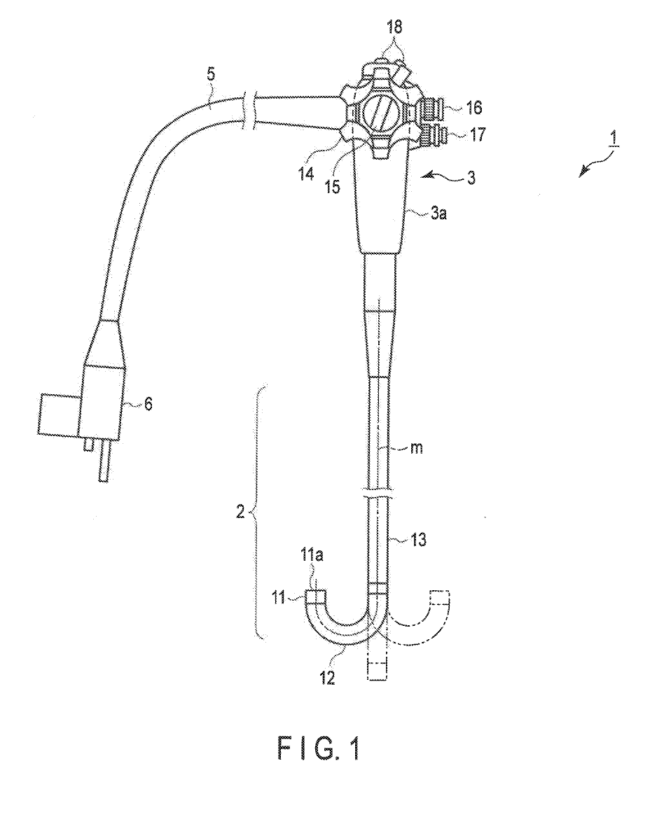

FIG. 1 is a diagram showing an outer appearance of an endoscopic main body according to the first embodiment.

The endoscope 1 includes a thin and long insertion section 2 that is to be inserted into a lumen, and an operation section 3 that is coupled to the proximal side of the insertion section 2 to manipulate the endoscope 1. The endoscope 1 according to the present embodiment may be applied to both an endoscope for observing living organs and a borescope for observing the interior of a metal pipe or internal combustion engine. In the embodiments described below, the resilience and initial tension are intended to mean as follows: The resilience denotes an “ability of a spiral tube to return to the original state (straight form) from a deformed state when an external force is applied”; and the initial tension denotes an “internal force (tightly attaching force) acting between the adjacent portions of an element wire that is spirally wound, without causing any deformation”. In the following explanation, the resilience and initial. tension are considered as acting similarly when an external force is applied.

The insertion section 2 mainly includes: a distal end portion 11, which is made of a hard member in which an imaging optical system and an illumination window of an imaging section are provided; a bendable portion 12 continuous with the proximal side of this distal end portion 11 to actively bend; and a soft flexible tube 13 continuous with the bendable portion 12 and connected to an operation section main body 3a of the operation section 3. Depending upon the purpose of use, the insertion section 2 may be provided separately with a forceps channel into which a treatment instrument is fit, passages for supplying and suctioning a cleaning liquid and air, and the like, in parallel inside the insertion section 2.

Openings for such a channel and passages are formed in the distal end surface 11a of the distal end portion 11.

The bendable portion 12 has a known structure in which a plurality of annular pieces (not shown) are coupled to one another, in a rotatable manner at their joints. The joints of the pieces are provided at alternatingly shifted positions so that any adjacent pair of joints are orthogonal to each other. A plurality of wires (not shown) connected to the pieces at the distal end are connected to angle knobs 14 and 15 provided in the operation section 3. When the angle knobs 14 and 15 are manipulated and rotated, the wires are pulled so that the bendable portion 12 can actively bend.

The operation section 3 includes an operation section main body 3a formed into a rectangular parallelepiped that can be easily held with one hand of the operator. With a universal cable 5 connected to the upper portion of the side face of the operation section 3, and the proximal side of the flexible tube 13 connected to the lower end of the operation section 3, the operation section 3 essentially forms an L-shape. The universal cable 5 may include an imaging/control signal cable, a power supply cable, a light guide for guiding illumination light, and the like, which are bundled together and covered with a resin coating member, although they are not shown. A connector terminal 6 is provided at the distal end. of the cable. The connector terminal 6 is connected at least to an image processing unit and a light source unit, which are not shown. As structural components of the system, the endoscope 1 also includes a monitor and an input device. The endoscope 1 may be provided with a pump unit which is used for supplying and suctioning air and water, treatment devices and the like, as needed.

On the front face of the operation section main body 3a, two angle knobs (14, 15) configured to bend the bendable portion 12 are coaxially arranged on top of the other. A suction switch 16 and an air/water supply switch 17 are Juxtaposed on the side face opposite to the side surface on which the universal cable 5 is provided, at positions comfortably reachable with the operator s fingers. On the top surface of the operation section main body 3a, photographing switches 18, including a shutter switch used for photographing an endoscopic image by the imaging optical system, are arranged.

The angle knobs 14 and 15 according to the present embodiment include a UD knob (first operation section) 14 which is rotated to bend the bendable portion 12 in the up/down direction (first axial direction), and an RL knob (second operation section) 15 which is rotated to bend the bendable portion 12 in the right/left direction (second axial direction) that is orthogonal to the first axial direction. In the present embodiment, manually-operable knobs are presented as an example. However, the angle knobs may also include motor switches for a bending operation using a driving source such as a motor.

The structure of the flexible tube 13 is now explained.

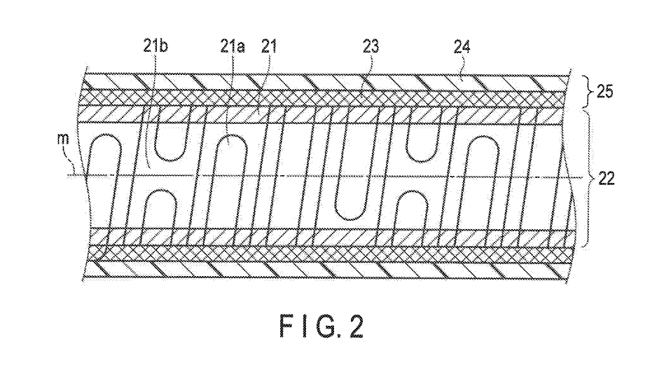

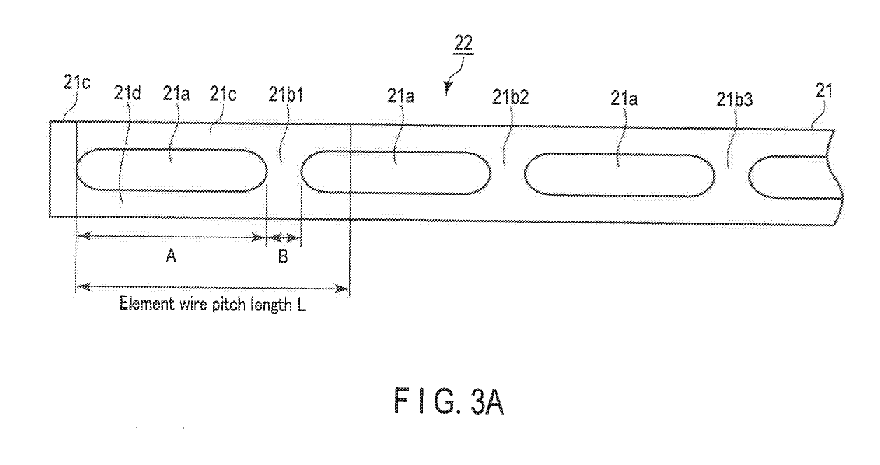

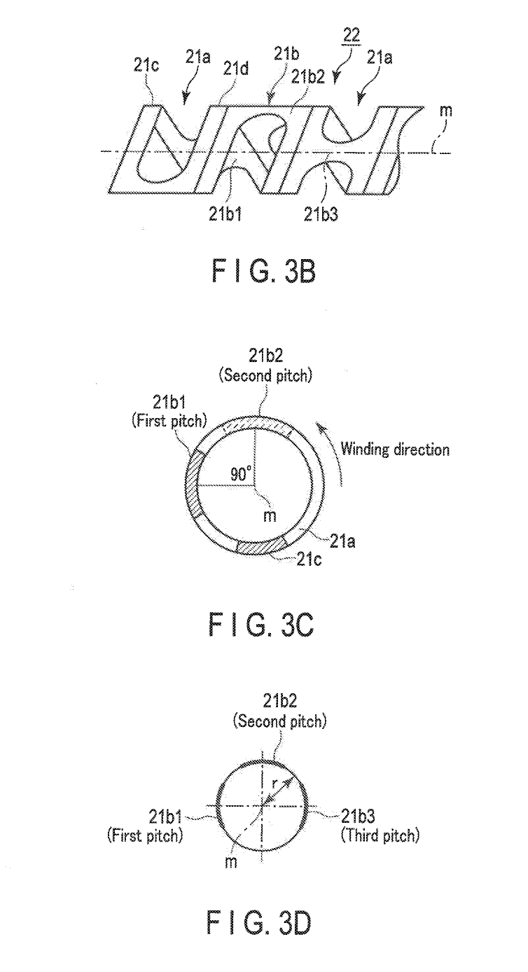

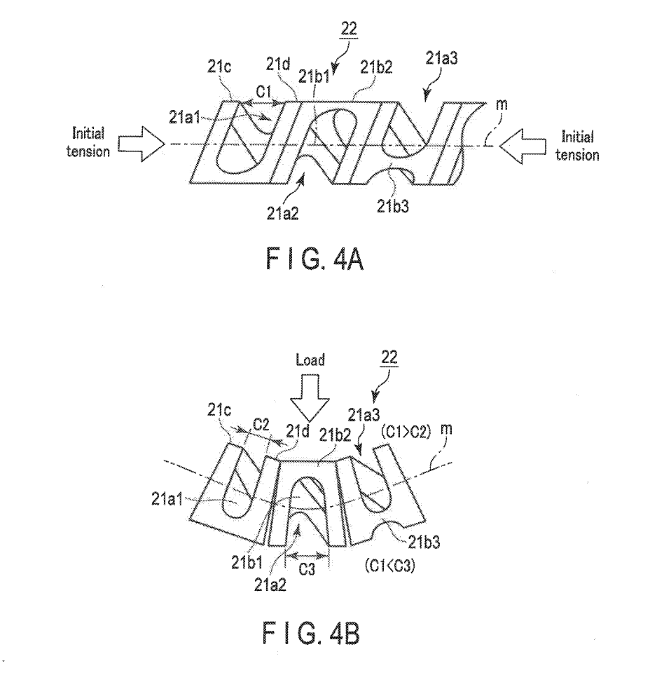

FIG. 2 is a sectional view of the flexible tube according to the present embodiment. FIG. 3A is a diagram showing an outer appearance of an element wire that forms a spiral tube when viewed from the above. FIG. 3B is a diagram showing an outer appearance of the spiral tube prepared by winding the element wire. FIG. 3C is a diagram showing the positional relationship of inter-hole portions (or retaining portions) at the first pitch and the second pitch. FIG. 3D is a conceptual diagram of the sectional structure of the spiral tube when viewed from the direction of the longitudinal axis. FIG. 4A is a diagram for showing the outer appearance of the spiral tube in the straight state, whereas FIG. 4B is a diagram for showing the spiral tube in the bent state.

In general, when the bendable portion 12 is inserted into a lumen, the flexible tube 13 is inserted continuously from the bendable portion 12, is bent suitably for the flexures of the lumen, and serves as a conveyor of a propulsion force to the inserted distal end portion 11.

As illustrated in FIG. 2, the flexible tube 13 has a hollow structure, inside which wires for a bending operation of the bendable portion 12, a light guide (optical fiber cable) for guiding illumination light, a signal cable for transmitting image capture signals, and the like are laid, although they are not shown. A forceps channel and water supply/suction passage (tube) may also be laid, in accordance with the design specification.

The flexible tube 13 has a multilayered structure including a spiral tube 22 that is provided inside and freely bendable, a reticulated tube 23 that covers the exterior surface of the spiral tube 22, and an outer sheath 24 that provides a water-tight covering for the exterior sur ace of the reticulated tube 23 and exhibits elasticity. The reticulated tube 23 and outer sheath 24 form an outer layer 25 together for the spiral tube 22. The spiral tube 22 that is covered with the outer layer 25 is fixed at its two ends so as not to change the overall length of the flexible tube. When the spiral tube 22 is in the straight state, an initial tension acts on the spiral tube 22 in the direction of the longitudinal axis m, as indicated in FIG. 4A.

For this spiral tube 22, an element wire 21 made of a metal member that is shaped into a long thin band-like plate as illustrated in FIG. 3A is used. As a metal member, a material used for a spring is suitable; for example, a material such as stainless steel and titanium steel that is corrosion-resistant and exhibits elasticity depending on its shape may be used, but is not constrained to these. The spiral tube 22 is formed by densely and spirally winding this element wire 21 so as not to create any space between the turns, as illustrated in FIGS. 3B and 4A.

Furthermore, the element wire 21 includes a plurality of oval adjustment portions 21a continuously in the longitudinal direction of the band, in predetermined intervals. The portions of the element wire 21 which are positioned on the two sides of an adjustment portion 21a in the width direction of the element wire 21 and have a predetermined width are referred to as edge portions 21c and 21d. This adjustment portion 21a may be either one of a through hole or a bottomed hole (a depression) such as a groove, as discussed later. Furthermore, an elastic material such as resin and rubber may be separately embedded into the hole 21a, or a film or the like may be adhered to cover the hole 21a.

When the element wire is wound, the adjustment portion 21a forms an opening (hole width C1 discussed later). In accordance with the degree of bending, the adjustment portion 21a adjusts the width of this opening by narrowing or widening the distance between the edge portions 21c and 21d with an inter-hole portion (or retaining portion) 21b serving as a fulcrum point. The change of the hole width from the bent state back to the straight state is realized by the elasticity. As illustrated in FIG. 4B, the inter-hole portion 21b2 serves as a fulcrum point, and at the same time it retains the hole width C1. The inter-hole portion 21b is therefore referred to as a retaining portion.

In the present embodiment, the hole 21a, which is a through-hole, will be discussed as an example of the adjustment portion 21a. In the element wire 21, holes 21a are continuously formed at intervals that are determined as the length of an inter-hole portion (retaining portion) 21b interposed between the holes 21a, These holes 21a are through-holes, which may be formed by cutting out with the laser processing or the like, or by perforating with the press working or the like. Preferably, the holes may be subjected to processing, including chamfering their corners by polishing or the like, so that stress would not be concentrated.

According to the present embodiment, the length AB obtained by adding the length A of the hole 21a to the length B of the inter-hole portion 21b is determined as 3/4 of the element wire pitch length L (i.e. the distance of one turn of the spiral winding). Because the spiral angle of the spiral tube 22 is small, the element wire pitch length L approximates the circumference 2πr of the spiral tube 22, where the radius of the spiral tube 22 is r, as illustrated in FIG. 3C. The length A of the hole 21a and the length B of the inter-hole portion 21b therefore can be expressed as A=7/6πr and B 1/3πr (1/6 of the circumference). That is, the length A of the hole 21a in the element wire 21 per pitch is greater than half the circumference of the spiral tube 22. In consideration of the arrangement in which the holes 21a are positioned on the two sides of the inter-hole portion 21b, the portions having a length greater than half the circumference of the spiral winding can serve as the edge portions 21c and 21d, which can be elastically deformed to widen or narrow the hole width C1.

In particular, as illustrated in FIG. 4B, when the spiral tube 22 is bent under a load, the holes 21a1 and 21a3 that have an opening (or are located) on the inner periphery side with respect to the longitudinal bending axis (central axis of the spiral tube 22) m are pushed by the not-shown portions of the element wire that are located on the two sides of these holes. Because of the hole 21a having the length A that is greater than half the circumference of the spiral tube 22, the edge portions 21c and 21d on the inner periphery side with respect to the longitudinal axis m are reliably deformed elastically and inwardly, with the element wire width retaining inter-hole portion 21b serving as a fulcrum point. The distance of the opening of each hole 21a is thereby reduced from the hole width C1 to the hole width C2 (C1>C2)

On the other hand, for the hole 21a2 having an opening (or located) on the outer periphery side, the edge portions 21c and 21d ate pulled by the portions of the element wire on the two sides and elastically deformed, with the inter-hole portion 21b serving as a fulcrum point. The opening is thereby increased from the hole width C1 to the hole width C3 (C3>C1) In the overall view of the spiral tube 22 that is deformed, the element wire width retaining inter-hole portion 21b is arranged at one position in each turn, which corresponds to the circumference of the spiral tube 22, to function as the fulcrum point, and the edge portions 21c and 21d that can be elastically deformed and have the length greater than half the circumference are provided on the two sides of the inter-hole portion 21b. With such an arrangement, the opening of the hole 21a positioned on the inner periphery side with respect to the bending is narrowed even under a small force, and the narrowed amount is absorbed by the hole 21a that is positioned on the outer periphery side and increases the width of its opening. On the other hand, when the hole 21a widens its opening, the corresponding length is absorbed by other holes by narrowing their opening. The length L of the spiral tube along the longitudinal axis m does not change before or after the bending.

Next, the bending radius R1 and the width C of the hole 21a and that can achieve the bendability suitable for the insertion when the spiral tube 22 is adopted for the flexible tube 13 explained.

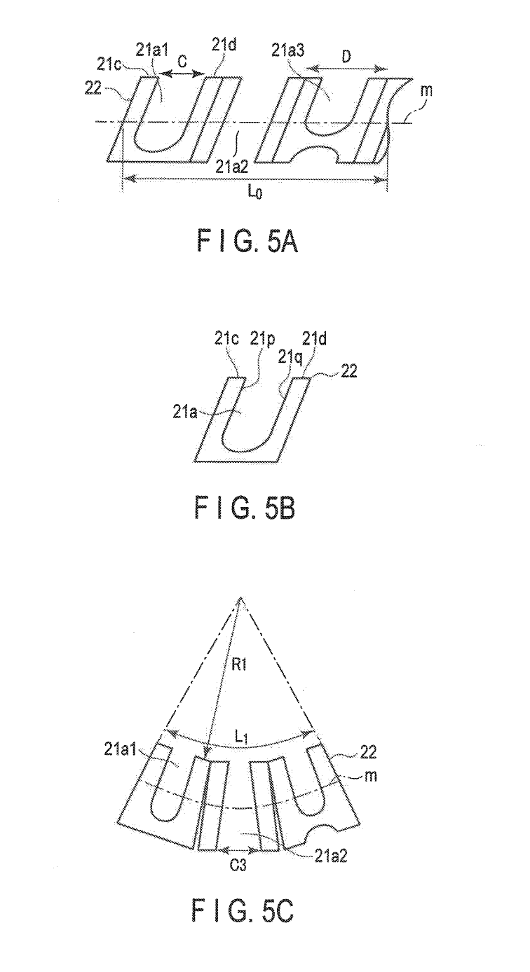

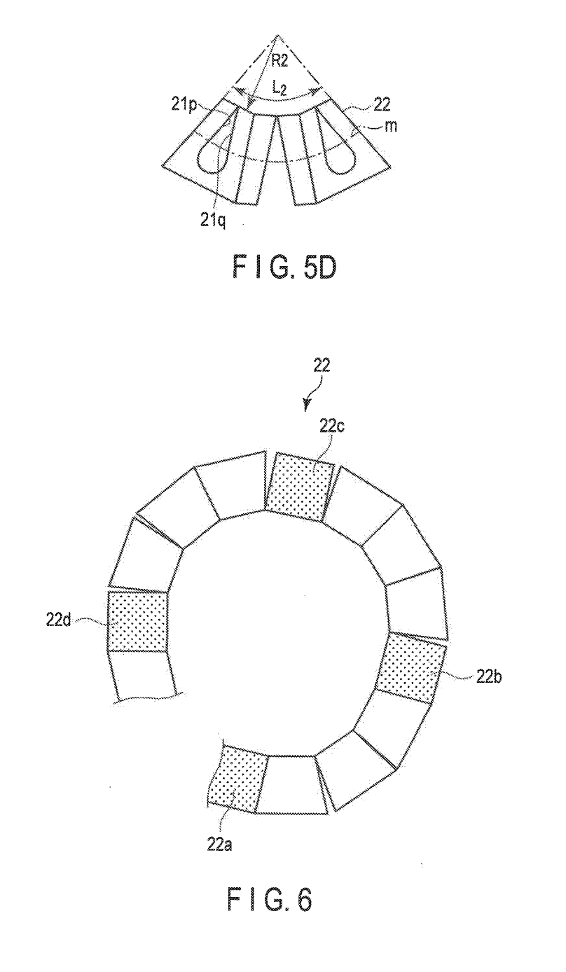

FIG. 5A is a conceptual diagram of the outer appearance of the spiral tube in the straight state; FIG. 5B is a diagram showing part of the structure of a hole in one pitch of the spiral tube; FIG. 5C is a conceptual diagram of the spiral tube in the state of being bent into a desired bending radius R1; and FIG. 5D is a conceptual diagram of the spiral tube in the state of being bent to the degree that the two walls of the hole are brought into contact. FIG. 6 is a conceptual diagram showing the positions of the inter-hole portions and the spiral tube in the bent state.

As illustrated in FIG. 5A, the spiral tube 22 is prepared by winding the element wire 21 having an element wire width D for the number n of turns, with a hole width C determined by the adjustment portion 21a, where the length of the bendable range in the longitudinal axis direction, or in other words, the length of the central axis, is L0. As illustrated in FIG. 5C, the inner peripheral length when being bent into a desired bending radius R1 is defined as L1. Similarly, as illustrated in FIG. 5D, the inner peripheral length is defined as L2 when being bent into the radius R2, where the hole width C1 reaches the narrowest point and the tips of the inner wall 21p and the inner wall 21q of the hole 21a are brought into contact.

According to the present embodiment, the element wire width D and the hole width C are determined to establish the relationship L2≦L1 between the inner peripheral lengths, while the longitudinal axis length L0 would not change after the bending. That is, the inner wall 21p and the inner wall. 21n of the hole 21a would not be brought into contact before the spiral tube 22 is bent into the desired bending radius R1 and the bending to the desired bending radius RI can be reliably achieved.

If an initial tension is applied to the spiral tube 22 in a structure in which the length of the spiral tube 22 includes a hole 21a having a length greater than one pitch in the direction of the longitudinal axis m and no inter-hole portion 21b is provided, the spiral tube 22 may not bear this initial tension, and may be deformed in the direction of the longitudinal axis m.

To address this, according to the present embodiment, the length AB, which is the sum of the length of the hole 21a and the length of the inter-hole portion 21b, is determined as 3/4L (=3/2πr) so that the inter-hole portions 21b are arranged in such a manner as illustrated in FIG. 3D by shifting the inter-hole portions 21b1 to 21b3, in this order, by 270 degrees in the circumferential direction around the longitudinal axis m of the spiral tube 22.

As a result, in the spiral winding, the inter-hole portions 21b are arranged by being shifted by 90 degrees between the first pitch and the second pitch as illustrated in FIG. 3C according to the present embodiment. Thus, no inter-hole portions (retaining portions) 21b in the adjacent portions of the element wire 21 would be arranged at the same position in the direction of the longitudinal axis. The inter-hole portions 21b are positioned every fourth pitch in line in the longitudinal direction, with three pitches in between, as illustrated in FIG. 6. With this arrangement, when viewed as the entire structure, the spiral tube 22 evenly bends. In the determination of the length AB, which is the sum of the length A and the length B, as 3/4L, one pitch of the element wire 21 always includes one inter-hole portion 21b. Because of this arrangement of one inter-hole portion 21b for one pitch, deformation is prevented even when an initial tension acts in the direction of the longitudinal axis m.

When a propulsion for insertion is applied from the proximal (operation section) side in the direction of the longitudinal direction, the spiral tube 22, if incorporated into the insertion section of the endoscope, can efficiently transmit this power to the distal (bendable portion) side. According to the present embodiment, the length AB is determined as A+B=3/4L, but should not be constrained to this limitation. As long as one inter-hole portion 21b is provided for one pitch of the element wire 21, and the adjacent portions of the element wire 21 that is wound are not positioned side by side in the direction of the longitudinal axis, the length AB is not limited.

As discussed above, even when bending occurs, the spiral tube of the present embodiment does not change its length in the direction of the longitudinal axis, a high initial tension can be applied, and a high resilience can be offered. In addition, in the spiral tube prepared by winding the element wire in which long oval holes are formed, the holes having the opening on the inner periphery side at the time of bending are narrowed at the opening, and the holes having the opening on the outer periphery side are widened at their opening. In this manner, the spiral tube can easily bend without changing its length in the direction of the longitudinal axis. The inter-hole portions, whose distance would not change even under a load from the adjacent portions of the element wire, are arranged by shifting their positions in the circumferential direction in such a manner that they would not be aligned next to each other in the direction of the longitudinal axis. Thus, holes are arranged on the two sides of each inter-hole portion. The entire spiral tube can therefore be uniformly bent and would not disturb the insertability into the body cavity. In other words, the retaining portions formed in the adjacent portions of the element wire wound in the spiral direction would not be positioned one next to another in the direction parallel to the longitudinal axis, but are shifted in the circumferential direction of the longitudinal axis.

### Second Embodiment

The second embodiment will be described.

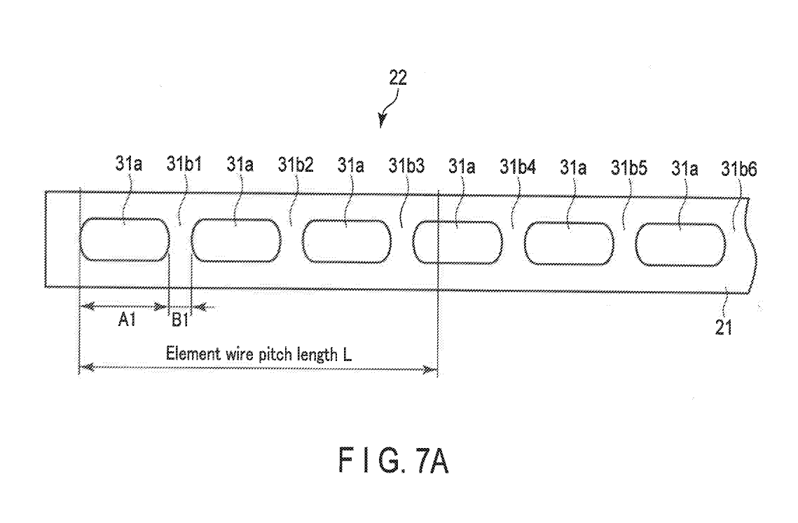

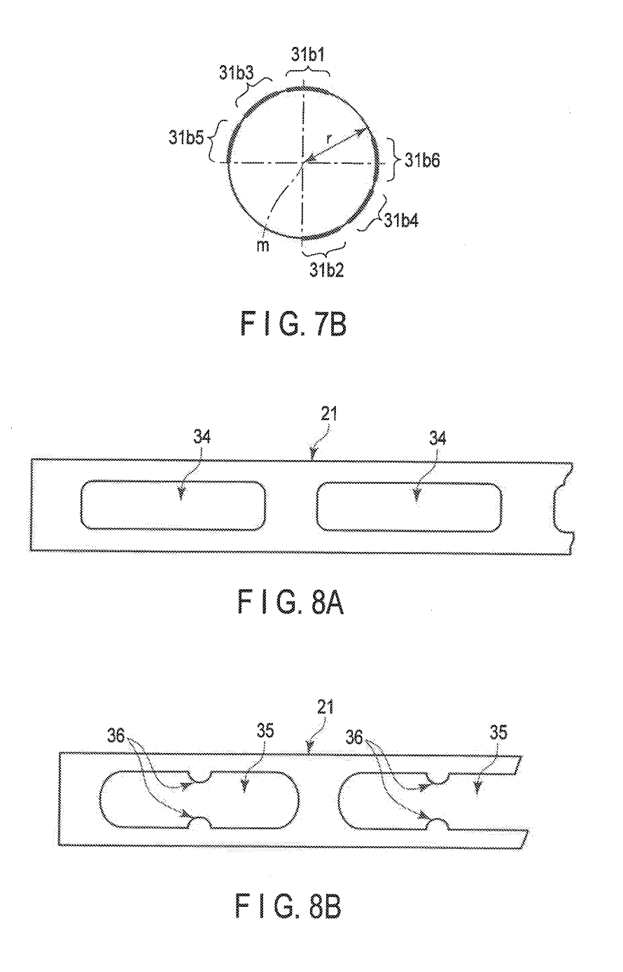

FIG. 7A is a diagram illustrating an outer appearance of the element wire that forms a flexible tube according to the second embodiment; and FIG. 7B is a conceptual diagram of the arrangement of inter-hole portions viewed from the direction of the longitudinal axis. According to the aforementioned first embodiment, one hole and one inter-hole portion are provided in one pitch. According to the present embodiment, a plurality of long oval holes 31a and inter-hole portions 31b are provided in one pitch. The structure other than the holes is the same as the aforementioned first embodiment, and thus the explanation of such a structure is omitted.

The inter-hole portions 31b as previously discussed are provided between these holes 31a. In FIG. 7A, inter-hole portions 31b1 to 31b6 are shown as examples. According to the present embodiment, for example, three times the length AB, which is the sum of the length A1 of the hole 31a and the length B1 of the inter-hole portion 31b, or 3(A1+B1), is determined as 5/6 of the element wire pitch length L.

3(A1+B1)=5/6 L

A1+B1=5/18L

This determines the length AB as 5/18L. Because the spiral angle of the spiral tube 22 is small, the element wire pitch length L can be approximated by the circumference 2πr of the spiral tube 22, where the radius of the spiral tube 22 is r. According to the present embodiment, when the length B1 is determined as:

B1=1/9πr(1/18 of the circumference)

the length A1 is determined as:

A1=7/18πr

The length A1 of the hole 31a and the length B1 of the inter-hole portion 31b can be defined as

3(A1+B1)=5/6L(=5/3πr)

This means that three inter-hole portions 31b are provided in one pitch of the element wire 21.

When the element wire 21 is spirally wound, the inter-hole portions 31b in the adjacent portions of the element wire 21 may partially overlap each other at their edges, but would not be provided next to each other in the direction of the longitudinal axis, as illustrated in FIG. 7B. According to the present embodiment, the arrangement of three holes in one pitch is discussed as an example of the arrangement of multiple holes, but the arrangement is not constrained thereto. As long as the inter-hole portions 31b in the adjacent portions of the element wire 21 are not next to each other in the direction of the longitudinal axis m, two or more inter-hole portions 31b may be arranged in one pitch of the element wire 21. The arrangement is not constrained to the above discussed lengths A1 and B1.

Because three or more inter-hole portions 31b are provided in one pitch of the element wire 21, the spiral tube 22 of the present embodiment is prevented from being tilted toward the bending direction or from leaking from the opening of a hole when force (initial tension or propulsion force for inserting the distal end portion 11) is applied in the direction of the longitudinal axis m. In comparison with the aforementioned first embodiment, this spiral tube is more resistant to deformation in the direction of the longitudinal axis, a still larger initial tension can be applied to the spiral tube, and a still higher resilience can be offered.

### First Modification

The first modification of the first and second embodiments will now be explained. FIG. 8A is a diagram showing the shape of holes according to the first modification. The element wire 21 according to the first and second embodiments discussed above are spirally wound and formed into a tube. When it is used, the element wire 21 is bent, and thus stress is produced.

The shape of a hole created in the element wire 21 is not constrained. The element wire 21 is a thin plate, and the opening of each hole is narrowed and widened when being bent. in consideration of plastic deformation and cracking, any concentration of stress resulting from the shape of the hole should preferably be avoided.

A hole 34 formed in the element wire 21 according to the present modification is shaped into a rectangle with its corners rounded. With the rounded corners, the concentration of stress produced can be avoided.

As the hole of the element wire 21 applicable to each of the above embodiments, it is preferable to provide a long hole that has a length of the opening (hole length L) so that the edge portions 21c and 21d can sufficiently present elastic deformation in accordance with the bending, and that has a combination of two longer sides that extend along the longitudinal direction and two shorter sides (hole width D) connecting these longer sides. If a semicircle is adopted on the shorter sides, an elliptic shape (or an oval track shape) can be obtained. In place of the semicircle, a polygon may be adopted on the shorter sides Oval, rhombic, or ovoidal shapes may also be applied to the embodiments and modification, as shapes having longer sides that are not straight.

### Second Modification

The second modification of the first and second embodiments will now be explained. FIG. 8B is a diagram showing the shape of holes according to the second modification.

The holes in the element wire of the spiral tube are configured so that the edge portions 21c and 21d are deformed in accordance with the bending of the spiral tube, narrowing, or widening the opening of the hole 21a. As discussed above, by suitably determining the element wire width D and the hole width C, a desired bending (curvature) can be achieved before the inner walls 21p and 21q come into contact.

In the examples of the flexible tubes 13 prepared for the endoscope 1 using the spiral tubes 22 of the previous embodiments, if an excessive load is accidentally applied and the flexible tube 13 is bent when the endoscope 1 is being the transported or sterilized, other than when used for observation, the inner walls 21p and 21g of the edge portions 21c and 21d of the element wire 21 may come into contact and push each other or collide with each other, causing plastic deformation.

For this reason, stoppers 36 are provided at or near the center of the inner side of the hole 35 to protrude and have an arc-shaped tip, as illustrated in FIG. 8B. These stoppers 6 avoid an unwanted contact of the inner walls of the hole 35, and particularly avoid plastic deformation which may be caused by the inner walls that are not merely in contact with each other, but that excessively overlap each other. In addition, with the arrangement of these stoppers 36, a large hole width (or a large opening) can be provided. In comparison with a hole with a smaller hole width, a structure with a higher bendability can be offered.

### Third Embodiment

The third embodiment is now described.

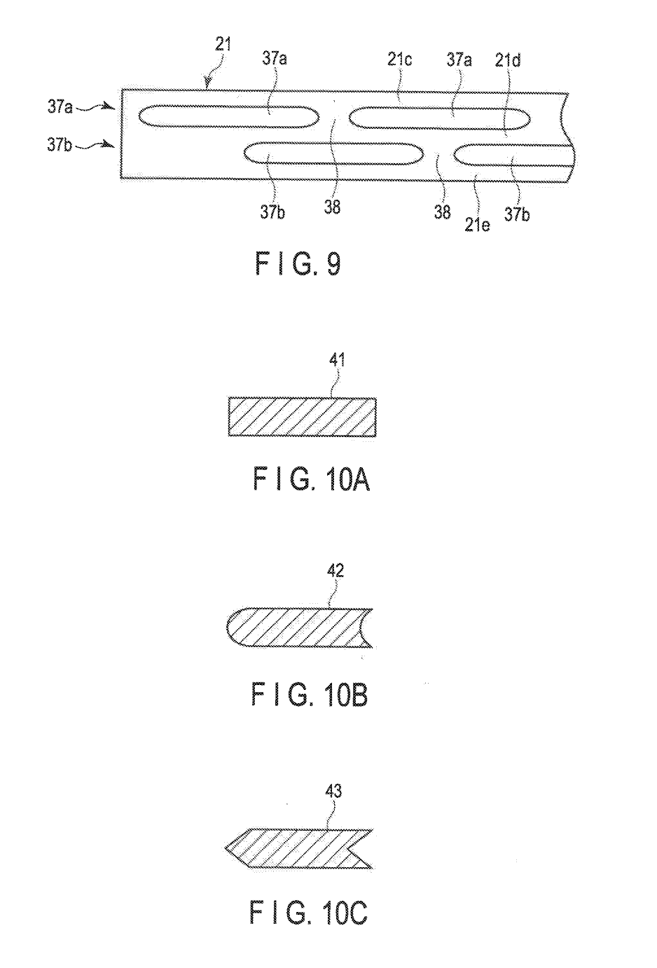

FIG. 9 is a diagram illustrating an outer appearance of an element wire that forms a flexible tube according to the third embodiment. According to the aforementioned first embodiment, holes are arranged in a single line in the longitudinal direction of the element wire 21. According to the present embodiment, holes are arranged in two rows in parallel to each other in the longitudinal direction of the element wire 21, with the holes and inter-hole portions provided in an alternating manner. The structure other than the holes is the same as the above-discussed first embodiment, and therefore the explanation is omitted.

As illustrated in FIG. 9, the holes 37a and 37b have the same shape and the same length, and are arranged in parallel in the same intervals along the longitudinal direction. The edge portion 21d is provided between these holes, and the inter-hole portions 38 are interposed between the holes 37a and between the holes 37b. Each of the inter-hole portions 38 in the first row is arranged so as to be next to the center position of the length of the hole 37b in the second row. The positions of the inter-hole portions 38 are shifted in the longitudinal direction so that they are not positioned side by side in the width direction of the element wire 21.

According to the preset embodiment, when such an element wire 21 is wound to form the spiral tube 22, the inter-hole portions 38 in each row are always adjacent to the holes 37a and 37b on the pitch. Thus, the opening of the holes on the inner periphery side of the spiral tube 22 is narrowed in whichever direction the spiral tube 22 is bent, making the spiral tube 22 easily bendable.

### Fourth Embodiment

The fourth embodiment will be now described.

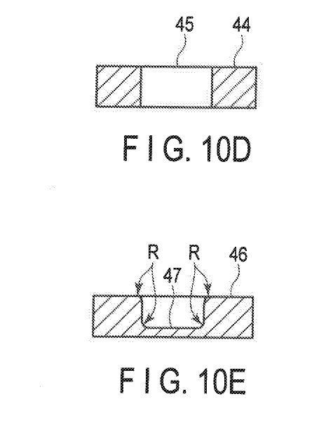

FIG. 10A shows a cross section of an element wire having a rectangular shape on the shorter sides, as the first example of the fourth embodiment. An element wire 41 in the first example can be easily processed.

FIG. 10B shows a cross section of an element wire having a convex curve and a concave curve on the shorter sides, as the second example of the fourth embodiment. When an element wire 42 of the second example is closely wound, the convex curve and the concave curve are brought into contact. As a result, when the spiral tube 22 is bent, the surfaces of the element wire slide each other without any displacement, and thus the spiral tube 22 can bend easily and continuously.

FIG. 10C shows a cross section of an element wire having a pointed end surface and an indented end surface, as the third example of the fourth embodiment. When an element wire 43 of the third example is closely wound, the pointed end surface and the indented end surface are brought into contact. As a result, when the spiral tube 22 is bent, the indented portion of the indented surface, with which the pointed end of the pointed surface is brought into contact, serves as a pivot point, and the surfaces of the portions of the element wire can thereby slide each other without displacement and further easily bend, forming a continuous curving surface.

FIG. 10D shows a cross section of an element wire haying a through-hole, as the fourth example of the fourth embodiment. This hole (through-hole) 45 is adopted in the above-discussed embodiments and modifications. The hole 45 can be perforated by a press-processing, and thus can be easily mass-produced at low cost. Preferably, the processing may be provided to chamfer the corners of the hole in the front and back surfaces so that the stress does not concentrate.

FIG. 10E shows a cross section of an element wire in which a bottomed groove (bottomed hole) 47 is formed as an adjustment portion in the element wire 46 in a manner not to penetrate the element wire 46, as the fifth example of the fourth embodiment. This groove 47 may be formed by laser processing (laser drawing) or etching. The groove 47 is also subjected to the rounding process to round the corners such as the corners determined by an inner wall and the top surface or corners determined by the inner wall and the bottom surface. The rounding process may be conducted at the time of forming the groove. The bottomed groove provided in place of the holes according to the present embodiment can produce similar effects to the above-discussed first and second embodiments.

The hole may be formed as a through-hole, and then a membrane such as a film may be adhered to the surface on the outer or inner side of the hole. Alternatively, the hole may be filled by an elastic material or resin material by insert molding or the like.

In the above-discussed embodiments and modifications, it is essential that the adjustment portions (holes or bottomed holes) are shaped so that the opening can move for closing and opening. The adjustment portions must have sides of suitable lengths in the longitudinal direction, or substantially in the longitudinal direction. For this reason, circular or triangular adjustment portions (holes and bottomed holes) are not suitable.

As the technique applicable to both the above-discussed embodiments and modifications, the element wire 21 is not constrained to the use of a single band-like member, and may be formed by combining different materials. In addition, the element wire 21 may be a single member or a complex of different members connected. The hardness processing does not have to be performed evenly, but the structure and processing may be changed in the width direction, in portions, or in the longitudinal direction.

The adjustment portions do riot have to be formed throughout the wound element wire, but the holes or bottomed holes may be formed partially in the range or positions where the spiral tube (or flexible tube) requires easy bending. The retaining portions (inter-hole portions 38) formed in a single element wire may not be formed in the same intervals, but adjustment portions may be formed to have different lengths and widths so that the spiral tube can be bent differently depending on its positions. The above-discussed embodiments show the entire element wire that is densely wound, as an example, but different winding manners may be combined. For example, loose winding may be applied to the area or positions on the proximal side that is not closely related to the insertion operation.

The present invention offers a flexible tube, in which the element wire of a flexible tube has elasticity in its width direction, is high in resilience and is easily bendable, as well as an endoscope that incorporates such a flexible tube. The above-discussed embodiments and modifications have the following functional effects:

(1) Because adjustment portions that are holes or bottomed holes are provided in the element wire of a densely wound spiral tube to which an initial tension is applied, the element wire on the inner peripheral side of the spiral tube can be elastically deformed and narrowed under a compressing force when the flexible tube is bent.

(2) Because an inter-hole portion, which serves as a retaining portion, is provided on each pitch of the element wire, the spiral tube is prevented from being deformed under an initial tension.

(3) An inter-hole portion arranged on the inner peripheral side of the spiral tube is difficult to be elastically deformed and difficult to bend. The inter-hole portions in the adjacent portions of the element wire of the spiral tube, however, are shifted some angle from each other in the peripheral direction of the spiral tube. The inter-hole portions therefore would not be positioned continuously in a certain peripheral direction of the spiral tube, but the openings of the holes are provided adjacent to the inter-hole portions. This allows the spiral tube to easily bend in any direction. Furthermore, because the inter-hole portions are arranged with a predetermined constant pitch, the entire spiral tube can he evenly bent.

(4) Because three or more inter-hole portions are provided in one pitch of the element wire, a large initial tension applied would not be absorbed by a change of the hole width. The shape of the spiral tube can thereby be retained.

(5) Holes are formed in two rows in the width of a single element wire in the longitudinal direction in such a manner that the inter-hole portion of the first hole portion is adjacent to the hole of the second hole portion. The spiral tube therefore can be narrowed on the inner peripheral side in any direction that the spiral tube is bent.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1-5. (canceled)

6. A flexible tube comprising:
a spiral tube configured by densely winding a band-like element wire into spiral form, the element wire having a pair of edge portions in a width direction and a plurality of sets of an adjustment portion and a retaining portion that are formed in the element wire in a continuous manner in a longitudinal direction of the element wire, wherein each of adjustment portions including the adjustment portion is arranged between the pair of edge portions and configured to adjust a distance between the pair of edge portions in accordance with an external force applied, and each of retaining portions including the retaining portion is configured to retain the distance between the pair of edge portions; and
an external layer that covers an external periphery of the spiral tube,
wherein the retaining portions formed in adjacent portions of the element wire when being wound are shifted in a peripheral direction of the longitudinal axis of the spiral tube so as not to be positioned continuously in a direction parallel to the longitudinal axis.

7. The flexible tube according to claim 6, wherein the adjustment portions formed in the element wire are holes or bottomed holes having a predetermined length in the edge portions.

8. The flexible tube according to claim 6, wherein at least one retaining portion is formed within a pitch of the element wire by adjusting a length of the adjustment portion, where the pitch is defined as a length of one turn of the winding.

9. The flexible tube according to claim 6, wherein: the adjustment portions and the retaining portions are alternatingly formed in two parallel rows along the longitudinal direction within a width of the element wire, and
the retaining portions are formed by shifting positions thereof in such a manner that the retaining portions in one row are not positioned side by side with the retaining portions in the other row.

10. The flexible tube according to claim 6, wherein the spiral tube is configured by applying an initial tension to the element wire along the direction of the longitudinal axis.

11. An endoscope comprising:
an operation section manipulated by an operator, and
an insertion section connected to the operation section and including the flexible tube as defined in claim 6.
